(19) United States
(12) Patent Application Publication
(10) Pub. No.: US 2008/0312300 A1
(43) Pub. Date: Dec. 18, 2008

(54) Processes for the Preparation of Stable Polymorphic Form I of Ritonavir

(76) Inventors: Yoginder Pal Sachdeva, Abohar (IN); Prosenjit Bose, Gurgaon (IN)

Correspondence Address: RANBAXY INC., 600 COLLEGE ROAD EAST, SUITE 2100, PRINCETON, NJ, 08540, US

(21) Appl. No.: 11/916,158

(22) PCT Filed: May 30, 2006
(86) PCT No.: PCT/IB06/51723

§ 371 Date: Jul. 9, 2008

(30) Foreign Application Priority Data
May 30, 2005 (IN) 1385/DEL/2005

(51) Int. Cl. C07D 417/12; A61K 31/427

(52) U.S. Cl. 514/365; 548/204

(57) ABSTRACT

The invention relates to processes for the preparation of a polymorphic form of ritonavir. More particularly, it relates to the preparation of a stable polymorphic Form I of ritonavir. The invention also relates to pharmaceutical compositions that include the stable Form I of ritonavir and use of the compositions for treatment of HIV infections in combination with other antiretro viral agents.

### FIELD OF THE INVENTION

The field of the invention relates to processes for the preparation of a polymorphic form of ritonavir. More particularly, it relates to the preparation of a stable polymorphic Form I of ritonavir. The invention also relates to pharmaceutical compositions that include the stable Form I of ritonavir and use of the compositions for treatment of HIV infections in combination with other antiretroviral agents.

### BACKGROUND OF THE INVENTION

Ritonavir of Formula I is chemically, [5S-(5R*,8R*,10R*,11R*)]-10-Hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester and is indicated in combination with other anti-retroviral agents for the treatment of HIV-infections.

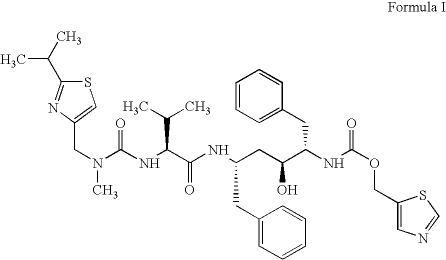

Several processes have been reported for the preparation of ritonavir for example, in U.S. Pat. Nos. 5,541,206 and 5,567,823; and International (PCT) Publication No. WO 00/04016.

International (PCT) Publication No. WO 00/04016 discloses processes for the preparation of crystalline Form II and amorphous forms of ritonavir. The disclosure of the '016 application suggests that the product obtained as per the processes of the '206 patent and the '823 patent is Form I of ritonavir. The disclosure further suggests that the Form I of ritonavir obtained as per the '206 and the '823 patent have more than 5 or 10% of Form II in it. Further it also provides a process for the preparation of Form I of ritonavir from Form II of ritonavir wherein the process involves the use of seed crystals of Form I of ritonavir.

U.S. Patent Application No. 2004/0024031 discloses Form III, Form IV and Form V of ritonavir and provides processes for their preparation. It also provides processes for the preparation of Form I and Form II of ritonavir.

Organic Process Research & Development 2000, 4:413-417 discloses processes for the preparation of Form I of ritonavir with less than 3% Form II of ritonavir. It also discloses a process for the preparation of Form II of ritonavir.

### SUMMARY OF THE INVENTION

The present inventors have noticed that Form I of ritonavir when prepared as per the process reported in the prior art is not stable and has a tendency to convert to other polymorphic forms upon storage, especially Form II of ritonavir. There is a significant difference in the solubility of Form I and Form II in water or dissolution media and Form II is not desirable form because of less solubility. The conversion of Form I to Form II results in difficulties for formulators because the quantities of excipients required for dissolving the bulk powder for preparation of a formulation vary significantly.

The present inventors have now obtained a stable polymorphic Form I of ritonavir having no or little tendency to convert to any other polymorphic form of ritonavir.

Accordingly, in one general aspect there is provided a stable polymorphic Form I of ritonavir. Embodiments of the stable polymorphic Form I of ritonavir may include one or more of the following features. For example, the ritonavir may have no detectable quantity of other polymorphic forms of ritonavir. The ritonavir may have 2% or less of Form II of ritonavir. The ritonavir may be incorporated into a dosage form with one or more pharmaceutically acceptable excipients. The ritonavir may have the XRD pattern illustrated in FIGS. 1 and/or 2.

In another general aspect there is provided a process for the preparation of Form I of ritonavir. The process includes obtaining a solution of ritonavir in one or more organic solvents; concentrating the solution to get a residue; adding an anti-solvent to the residue; and isolating the Form I of ritonavir by the removal of the solvents.

Embodiments of the process may include one or more of the following features. For example, the anti-solvent may be characterized by the ritonavir being insoluble, practically insoluble or very slightly soluble in the anti-solvent.

Removing the solvents may include, for example, one or more of filtration, filtration under vacuum, decantation and centrifugation. The process may include further forming of the product so obtained into a finished dosage form.

The process may include further drying of the product obtained.

In another general aspect there is provided a pharmaceutical composition that includes a therapeutically effective amount of the Form I of ritonavir; and one or more pharmaceutically acceptable carriers, excipients or diluents.

In another general aspect there is provided a method for treating HIV-1 infections in a warm-blooded animal. The method includes providing a pharmaceutical composition to the warm-blooded animal, the pharmaceutical composition comprising Form I of ritonavir.

The details of one or more embodiments of the invention are set forth in the description below. Other features, objects and advantages of the invention will be apparent from the description and claims.

### DESCRIPTION OF THE DRAWINGS

FIGS. 1a and 1b are an X-ray powder diffraction pattern of polymorphic Form I of ritonavir and the associated values, respectively, prepared as per Example 1.

FIGS. 2a and 2b are an X-ray powder diffraction pattern of polymorphic Form I of ritonavir and the associated values, respectively, prepared as per Example 2.

FIGS. 3a and 3b are an X-ray powder diffraction pattern of polymorphic Form I of ritonavir and the associated values, respectively, prepared as per Example 2 stored at 25° C. after 3 months.

### DETAILED DESCRIPTION OF THE INVENTION

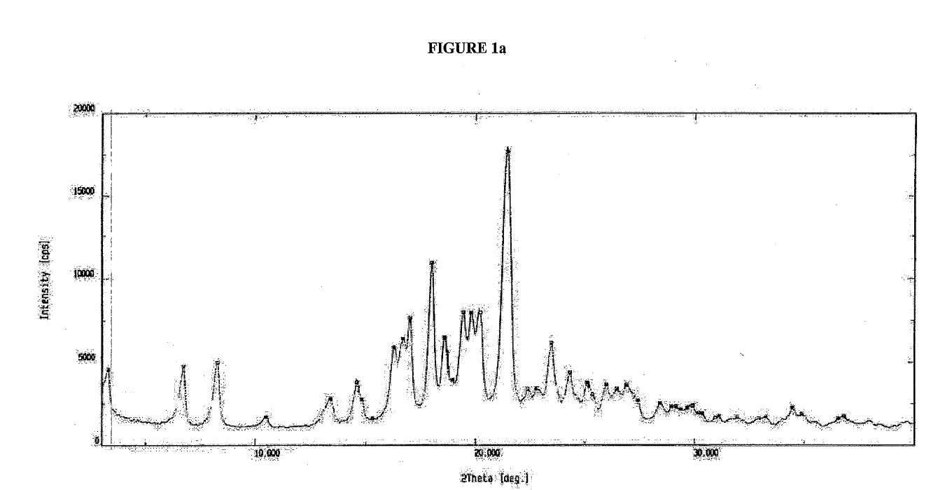

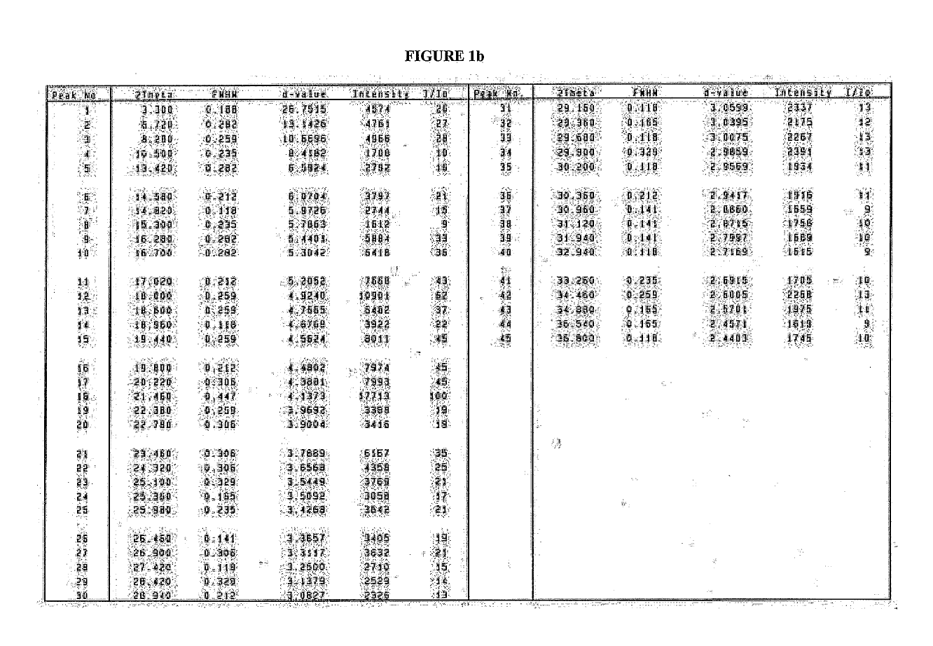

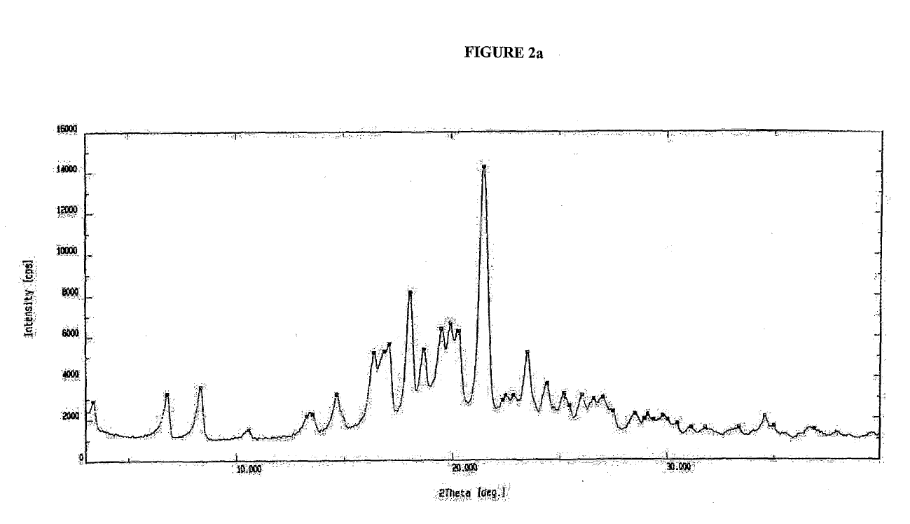

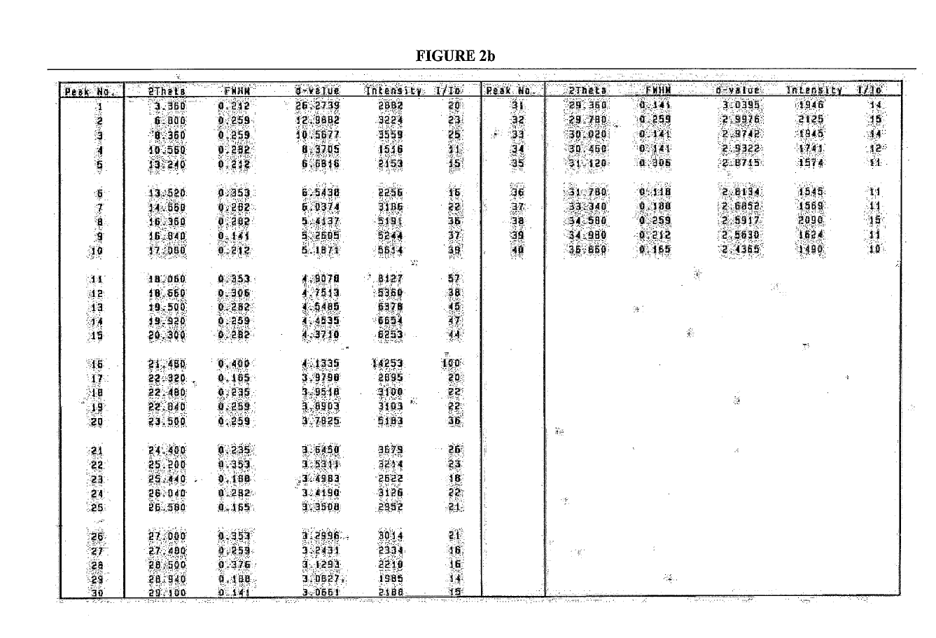

A first aspect of the present invention provides stable polymorphic Form I of ritonavir having no tendency to convert to any other polymorphic form. The stable polymorphic Form I of ritonavir of the present invention has 2% or less of Form II of ritonavir. More preferably the stable Form I of ritonavir has no detectable quantity of Form II of ritonavir. The X-Ray powdered Diffraction (XRPD) pattern of stable Form I of ritonavir is provided in FIGS. 1a and 2a of the drawing. The stability study performed on stable Form I of ritonavir suggests that it is stable under normal storage conditions.

A second aspect of the invention provides a process for preparing stable Form I of ritonavir. The process includes the steps of:

a) obtaining a solution of ritonavir in one or more organic solvents;

b) concentrating the solution to get a residue;

c) adding an anti-solvent to the residue; and

d) isolating the Form I of ritonavir by the removal of the solvents.

The present inventors have found that no seed crystals are required for preparing the Form I.

Ritonavir to be used as the starting material can be prepared by any process known in the literature for example, in U.S. Pat. Nos. 5,541,206 and 5,567,823; and International (PCT) Publication No. WO 00/04016. The so-obtained ritonavir is suspended in an organic solvent and a solution of ritonavir is obtained. The solution may be obtained by heating the ritonavir in an organic solvent. The resultant solution can be clarified to remove foreign particulate matter or treated with activated charcoal to remove coloring and other related impurities. The solution so obtained may be concentrated to reduce the amount of solvent. The solution may be concentrated by removing the solvent completely to get a residue. The solvent may be removed under reduced pressure. To the residue so obtained an anti-solvent is added. The anti-solvent is characterized by the fact that ritonavir is insoluble, practically insoluble or very slightly soluble in the anti-solvent. The terms insoluble, practically insoluble and very slightly soluble have their ordinary meanings as defined in United States Pharmacopoeia 2002.

The term “suitable solvents” includes any solvent or solvent mixture in which ritonavir can be solubilized, including, for example, esters; lower alkanols; ethers; ketones; polar aprotic solvents, halogenated hydrocarbons, or mixtures thereof.

The esters may include one or more of ethyl acetate, n-propyl acetate, isopropyl acetate, and n-butyl acetate. Examples of alkanol include those primary, secondary and tertiary alcohols having from one to six carbon atoms. Suitable lower alkanol solvents include methanol, ethanol, n-propanol, isopropanol and butanol. Examples of chlorinated hydrocarbons include dichloromethane, chloroform, and 1,2-dichloroethane. Examples of ketones include acetone, methyl ethyl ketone and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone. Mixtures of all of these solvents are also contemplated.

A suitable anti-solvent that may be added to precipitate out Form I of ritonavir includes C₅₋₇ straight or branched chain alkanes, C₅₋₇ cycloalkanes, C₄₋₁₂ ethers, petroleum ether, and mixtures thereof. The reaction mass can be stirred for some time for example, from about 10 minutes to about 6 hours to get Form I of ritonavir. The solvent may be removed from the solution by a technique which includes, for example, filtration, filtration under vacuum, decantation and centrifugation. The product may be washed and dried by conventional methods.

A third aspect of the present invention provides a pharmaceutical composition comprising as its active ingredient stable polymorphic Form I of ritonavir having no tendency to convert to any other polymorphic form. With the active ingredient, the pharmaceutical composition includes one or more pharmaceutically acceptable excipients/diluents. The pharmaceutical composition of the present invention may be in the form of a solid or liquid dosage forms for oral, parenteral or topical use and may have immediate or sustained release characteristics. The dosage forms possible include tablets, capsules, powders, granules, creams, lotions, ointments, injectables, ophthalmic or otic solutions, suspensions, elixirs and the like.

A fourth aspect of the present invention provides a method of treating HIV infections by administering to a mammal in need thereof a therapeutically effective amount of stable polymorphic Form I of ritonavir having no tendency to convert to any other polymorphic form.

While the present invention has been described in terms of its specific embodiments, certain modifications and equivalents will be apparent to those skilled in the art and are intended to be included within the scope of the present invention.

### EXAMPLE 1

### Preparation of Form I of Ritonavir

Ritonavir (5.0 g) was suspended in ethyl acetate (37.5 ml). The mixture was stirred and heated at 60° C. till the entire solid dissolved. The solution was filtered to remove any undissolved suspended particles. The filtrate was concentrated under vacuum at 60° C. completely to give an oily residue, which was cooled at 30° C. and n-heptane (50 ml) was charged. The contents were stirred for 16-17 hours at 30° C. N-heptane (50 ml) was added to the thick slurry so obtained and stirred for another 4 hours at 30° C. The solid was filtered and dried under vacuum at 60° C. for 24 hours.

Yield: 4.5 g

### EXAMPLE 2

### Preparation of Form I of Ritonavir

Ritonavir (0.85 kg) was suspended in ethyl acetate (7.5 l). The mixture was stirred and heated at 60° C. till the entire solid dissolved. The solution was filtered to remove any undissolved suspended particles and washed with ethyl acetate (0.5 l). The filtrate was concentrated under vacuum at 60° C. completely to give an oily residue, which was cooled at 30° C. and n-heptane (8.5 l) was charged. The contents were stirred for 18 hours at 30° C. N-heptane (8.5 l) was added to the thick slurry so obtained and stirred for another 3-4 hours at 30° C. The solid was washed with n-heptane (1.7 l) filtered and dried under vacuum at 60° C. for 24 hours to obtain the title compound having the XRD pattern depicted in FIG. 2.

Yield: 0.82 kg

Form II: Not detectable

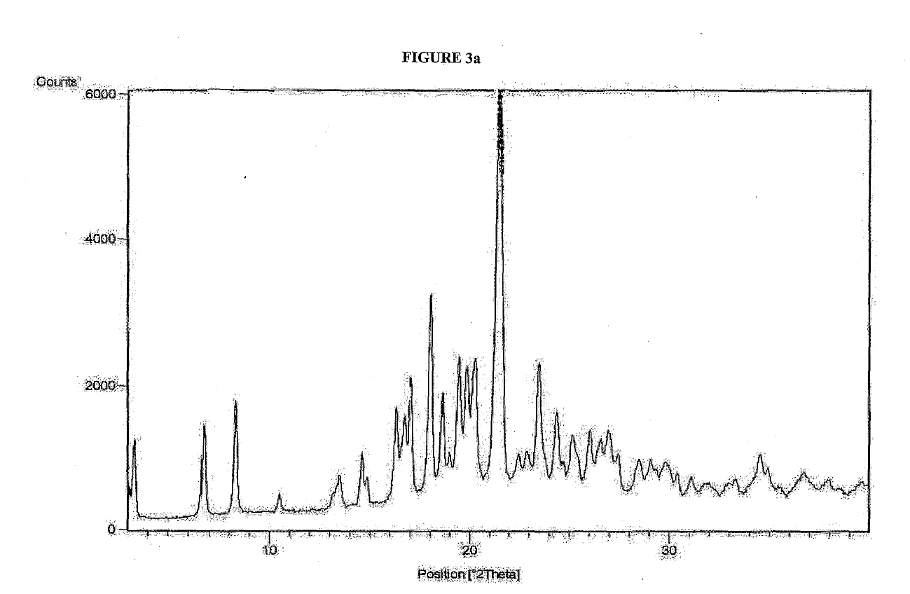

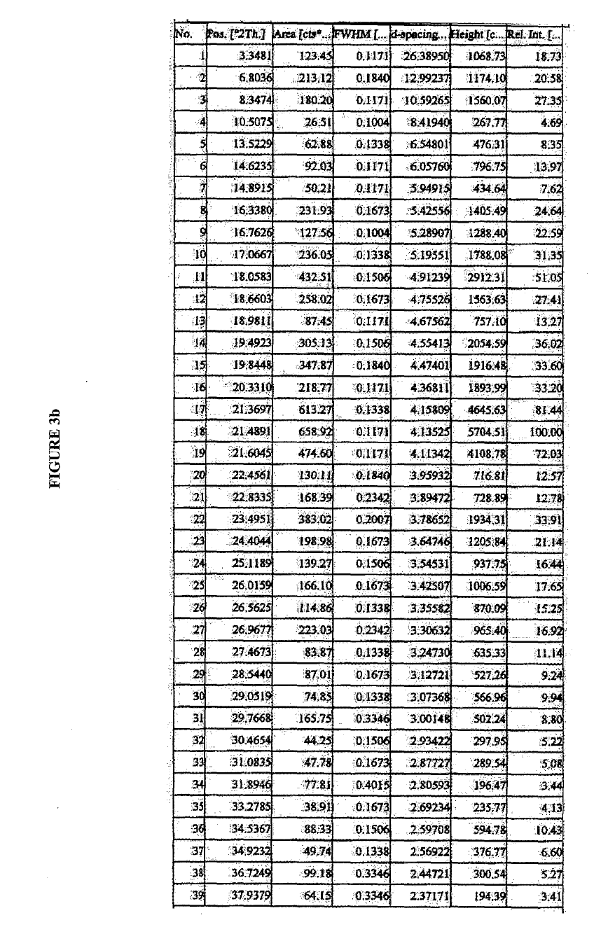

Stability Data The product obtained as per Example 2 was stored at 25° C. for a period of 3 months and no conversion in the polymorphic form was observed. (XRD pattern of the title compound stored at 25° C. after 3 months is depicted in FIG. 3).

While the present invention has been described in terms of its specific embodiments, certain modifications and equivalents will be apparent to those skilled in the art and are intended to be included within the scope of the present invention. For example, the polymorphic forms described herein can be formulated into dosage forms that are suitable for administering to patients in need of the compound for treating a medical condition for which the rotinavir is indicated, approved, or otherwise beneficial. Specifically, the Forms I of ritonavir can be formulated with one or more pharmaceutically acceptable excipients and/or with one or more active ingredients into a dosage form and administered to treat HIV infections.

1. A process for the preparation of Form I of ritonavir, the process comprising:
e) obtaining a solution of ritonavir in one or more organic solvents;
f) concentrating the solution to get a residue;
g) adding an anti-solvent to the residue; and
h) isolating the Form I of ritonavir by the removal of the solvents.

2. The process according to claim 1, wherein the organic solvent comprises one or more of esters, lower alkanols, ethers, ketones, polar aprotic solvents, halogenated hydrocarbons, or mixtures thereof.

3. The process according to claim 2, wherein the ester comprises one or more of ethyl acetate, n-propyl acetate, isopropyl acetate, and n-butyl acetate.

4. The process according to claim 3, wherein the ester is ethyl acetate.

5. The process according to claim 2, wherein the lower alkanol comprises one or more of methanol, ethanol, n-propanol, isopropanol, butanol and isobutanol.

6. The process according to claim 2, wherein the ether comprises one or both of diethyl ether and tetrahydrofuran.

7. The process according to claim 2, wherein the ketone comprises one or both of acetone, and methyl ethyl ketone.

8. The process according to claim 2, wherein the polar aprotic solvent comprises one or more of N,N-dimethylformamide, N,N-dimethylacetamide, dimethylsulfoxide, acetonitrile, and N-methylpyrrolidone.

9. The process according to claim 2, wherein the halogenated hydrocarbon comprises one or more of dichloromethane, chloroform and 1,2-dichloroethane.

10. The process according to claim 1, wherein the anti-solvent comprises one or more of C₅₋₇ straight or branched chain alkanes, C₅₋₇ cycloalkanes, C₄₋₁₂ ethers, petroleum ether, or mixtures thereof.

11. The process according to claim 1, wherein removing the solvent comprises one or more of filtration, filtration under vacuum, decantation and centrifugation.

12. The process according to claim 1, further comprising additional drying of the product obtained.

13. The process according to claim 1, wherein the Form I of ritonavir has the X-ray diffraction pattern of FIG. 1.

14. Storage stable Form I of ritonavir.

15. The stable Form I of ritonavir according to claim 14, wherein the ritonavir has no detectable quantity of Form II of ritonavir after 3 months of storage at 25° C.

16. The stable Form I of ritonavir according to claim 14, wherein the ritonavir has 2% or less of Form II of ritonavir.

17. The stable Form I of ritonavir of claim 14, wherein the ritonavir has the X-ray diffraction pattern of FIG. 3.

18. A pharmaceutical composition comprising a therapeutically effective amount of a storage stable Form I of ritonavir having no detectable quantity of Form II of ritonavir, and one or more pharmaceutically acceptable carriers, excipients or diluents.
